(19) United States
(12) Patent Application Publication
(10) Pub. No.: US 2019/0298150 A1
(43) Pub. Date: Oct. 3, 2019

(54) CONTROL DEVICE AND METHOD, PROGRAM, AND ENDOSCOPE SYSTEM

(71) Applicant: Sony Corporation, Tokyo (JP)

(72) Inventors: Takeshi MIYAI, Kanagawa (JP); Hisakazu SHIRAKI, Kanagawa (JP); Tadashi MORISHIGE, Kanagawa (JP); Kentaro FUKAZAWA, Tokyo (JP)

(73) Assignee: Sony Corporation, Tokyo (JP)

(21) Appl. No.: 16/306,965

(22) PCT Filed: Jun. 13, 2017
(86) PCT No.: PCT/JP2017/021733

§ 371 Date: Dec. 4, 2018

(30) Foreign Application Priority Data
Jun. 27, 2016 (JP) 2016-126298

(51) Int. Cl. A61B 1/00; G02B 7/36; G02B 23/24

(52) U.S. Cl. CPC A61B 1/00006; G02B 7/36; A61B 1/0676; A61B 1/00188; G02B 23/24

(57) ABSTRACT

The present disclosure relates to a control device and method, a program, and an endoscope system that are designed to be capable of shortening of the time before focusing.

A target point distance calculation unit acquires an illumination intensity from an AE control unit, acquires a target point luminance value from a target point determination unit, and acquires an object reflectance from a reflectance holding unit. The target point distance calculation unit calculates (predicts) the distance to a target point, using the illumination intensity acquired from the AE control unit, the target point luminance value acquired from the target point determination unit, and the object reflectance acquired from the reflectance holding unit. Upon receipt of the target point distance from the target point distance calculation unit, the AF control unit searches for the focus position, using the received target point distance as the initial value. The present disclosure can be applied to an endoscope system to be used for surgeries, examinations, and the like, for example.

### TECHNICAL FIELD

The present disclosure relates to a control device and method, a program, and an endoscope system, and more particularly, to a control device and method, a program, and an endoscope system that are designed to be capable of shortening the time before focusing.

### BACKGROUND ART

In a contrast automatic focus (AF) process, a long period of time is required before focus is achieved (see Patent Document 1).

### CITATION LIST

### Patent Document

Patent Document 1: Japanese Patent Application Laid-Open No. 2009-142586

### SUMMARY OF THE INVENTION

### Problems to be Solved by the Invention

In view of the above, there is a demand for a higher-speed contrast AF process.

The present disclosure is made in view of those circumstances, and aims to shorten the time before focusing.

### Solutions to Problems

A control device according to one aspect of the present technology includes: a distance calculation unit that calculates a distance from a lens to a target point in an image, using luminance information about the target point, a reflectance of the target point, and an intensity of a light, the lens having a fixed positional relationship with the light; and a focus control unit that searches for a focus position, with an initial value being the distance to the target point calculated by the distance calculation unit.

The control device may further include a target point determination unit that determines whether or not the target point is a new target point. In a case where the target point determination unit determines that the target point is not a new target point, the distance calculation unit calculates a distance to the target point, and the focus control unit searches for the focus position, with the initial value being the distance to the target point calculated by the distance calculation unit.

The control device may further include a reflectance calculation unit that calculates the reflectance of the target point. In a case where the target point determination unit determines that the target point is a new target point, the focus control unit searches for the focus position to acquire a distance to the target point, and the reflectance calculation unit calculates the reflectance of the target point, using the distance to the target point acquired by the focus control unit, the luminance information about the target point, and the intensity of the light.

The reflectance of the target point calculated by the reflectance calculation unit is registered in association with target point information about the target point.

The reflectance of the target point is registered in association with the target point information about the target point.

The reflectance of the target point is registered for each organ.

The reflectance of the target point is registered for each individual.

The target point determination unit may determine whether or not the target point is a new target point, by matching.

The control device may further include a position information transmission unit that transmits position information to a mobile object, the position information being for moving the moving object to a position to which the user is paying attention as determined by the attention determination unit.

The control device may further include a contrast determination unit that determines whether or not a contrast ratio in the image is lower than a threshold value. In a case where the contrast determination unit determines that the contrast ratio in the image is lower than the threshold value, the distance calculation unit calculates a distance to the target point, and the focus control unit searches for the focus position, with the initial value being the distance to the target point calculated by the distance calculation unit.

In a case where the contrast determination unit determines that the contrast ratio in the image is higher than the threshold value, the focus control unit can search for the focus position without the initial value.

The lens and the light are provided in an endoscope.

A control method according to the one aspect of the present technology is implemented by a control device, and includes: calculating a distance from a lens to a target point in an image, using luminance information about the target point, a reflectance of the target point, and an intensity of a light, the lens having a fixed positional relationship with the light; and searching for a focus position, with an initial value being the calculated distance to the target point.

A program according to the one aspect of the present technology causes a computer to function as: a distance calculation unit that calculates a distance from a lens to a target point in an image, using luminance information about the target point, a reflectance of the target point, and an intensity of a light, the lens having a fixed positional relationship with the light; and a focus control unit that searches for a focus position, with an initial value being the distance to the target point calculated by the distance calculation unit.

An endoscope system according to another aspect of the present technology includes: an endoscope including: a light that illuminates a subject; a lens that has a fixed positional relationship with the light; and an imaging unit that captures an image of a subject by capturing, via the lens, light reflected from the subject illuminated with the light; and a control device including: a distance calculation unit that calculates a distance from the lens to a target point, using luminance information about the target point in the image captured by the imaging unit, a reflectance of the target point, and an intensity of the light; and a focus control unit that searches for a focus position, with an initial value being the distance to the target point calculated by the distance calculation unit.

In one aspect of the present technology, the distance from a lens to a target point in an image is calculated from luminance information about the target point, the reflectance of the target point, and the intensity of a light, with the lens having a fixed positional relationship with the light. The focus position is then searched for, with the initial value being the calculated distance to the target point.

In another aspect of the present technology, light reflected from a subject illuminated with a light designed to illuminate a subject is captured by an endoscope via a lens having a fixed positional relationship with the light. In this manner, an image of the subject is captured. A control device calculates the distance from a lens to a target point in a captured image, using luminance information about the target point, the reflectance of the target point, and the intensity of a light, while the lens has a fixed positional relationship with the light. The control device then searches for the focus position, with the initial value being the calculated distance to the target point.

### Effects of the Invention

According to the present technology, it is possible to shorten the time before focusing.

Note that the advantageous effects described in this specification are merely examples, and the advantageous effects of the present technology are not limited to them and may include some additional effects.

### BRIEF DESCRIPTION OF DRAWINGS

FIG. 1 is a diagram showing an example configuration of an endoscope system of the present technology.

FIG. 2 is a block diagram showing example configurations of an endoscope and an endoscope control device.

FIG. 3 is a flowchart for explaining a high-speed contrast AF process to be performed by the endoscope system.

FIG. 4 is a diagram for explaining the process in a first stage and the data flow in the high-speed contrast AF process.

FIG. 5 is a diagram for explaining the process in a second stage and the data flow in the high-speed contrast AF process.

FIG. 6 is a flowchart for explaining a contrast AF process.

FIG. 7 is a block diagram showing an example configuration of the hardware of a personal computer.

### MODES FOR CARRYING OUT THE INVENTION

The following is a description of modes for carrying out the present disclosure (hereinafter referred to as embodiments).

### 1. First Embodiment

<Endoscope System of the Present Technology>

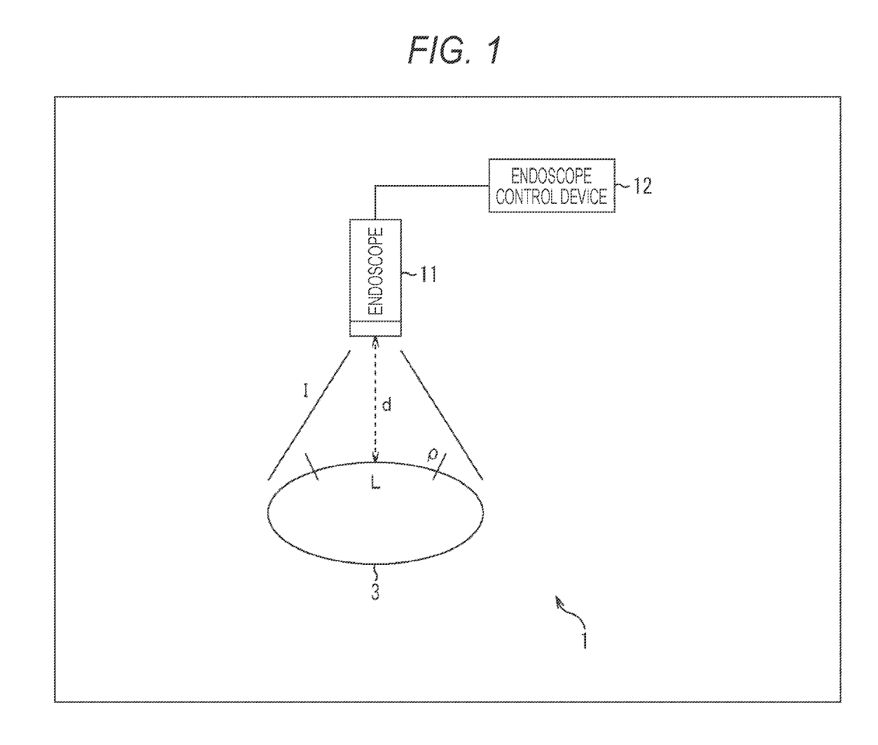

First, referring to FIG. 1, the principles of the present technology are described.

The example in FIG. 1 shows an endoscope system 1 to which the present technology is applied. The endoscope system 1 includes an endoscope 11 that captures an image of a subject 3 while illuminating the subject 3 with a light source (a light), and an endoscope control device 12 that controls the endoscope 11 and processes the image captured by the endoscope 11. Note that the endoscope 11 and the endoscope control device 12 are connected in a wired or wireless manner, for example, and communicate with each other.

Here, the example in FIG. 1 shows the following four values: the illumination intensity I [cd] for illuminating the subject 3 from the light of the endoscope 11, the distance d [m] from the light of the endoscope 11 to the target point, the object reflectance p of the subject 3, and the target point luminance L [cd/m\hat{ }2]. The relationship among these four values is expressed by the equation (1) shown below.

$$[ Mathematical Formula 1 ]$$

$$L = \frac{I ρ}{π {d}^{2}} \qquad ( 1 )$$

Note that π represents the circumference ratio.

That is, if three of the four values are acquired, the remaining one can be calculated. For example, the object reflectance p is expressed by the following equation (2).

$$[ Mathematical Formula 2 ]$$

$$ρ = \frac{L π {d}^{2}}{I} \qquad ( 2 )$$

Meanwhile, the distance d to the target point is expressed by the following equation (3).

$$[ Mathematical Formula 3 ]$$

$$d = \sqrt{\frac{I ρ}{L π}} \qquad ( 3 )$$

In the endoscope 11 or the like, the positional relationship between the objective lens and the light is fixed at the exit of emitted light. Accordingly, in a case where all components always move together, the distance from the light to the target point is apparent, as long as the distance from the lens to the target point is known. Note that the objective lens and the light are not necessarily located the tip. The distance from the lens to the target point is obtained when an automatic focus (AF) operation is performed to achieve focus. Conversely, if this distance is known in advance, it is possible to speed up the AF by using the known distance from the lens to the target point as the initial value of the AF.

That is, the process according to the present technology includes two stages. In the first stage, a normal AF operation is performed on a new target point, and the reflectance of the target point is acquired from the result. In the second stage, if the target point is the same as before, the reflectance is also the same. In view of this, the distance to the target point is estimated from the reflectance previously acquired, and an AF operation using the estimated distance as the initial value is performed. In this manner, the AF operation speed is increased.

<Configurations of the Endoscope and the Endoscope Control Device>

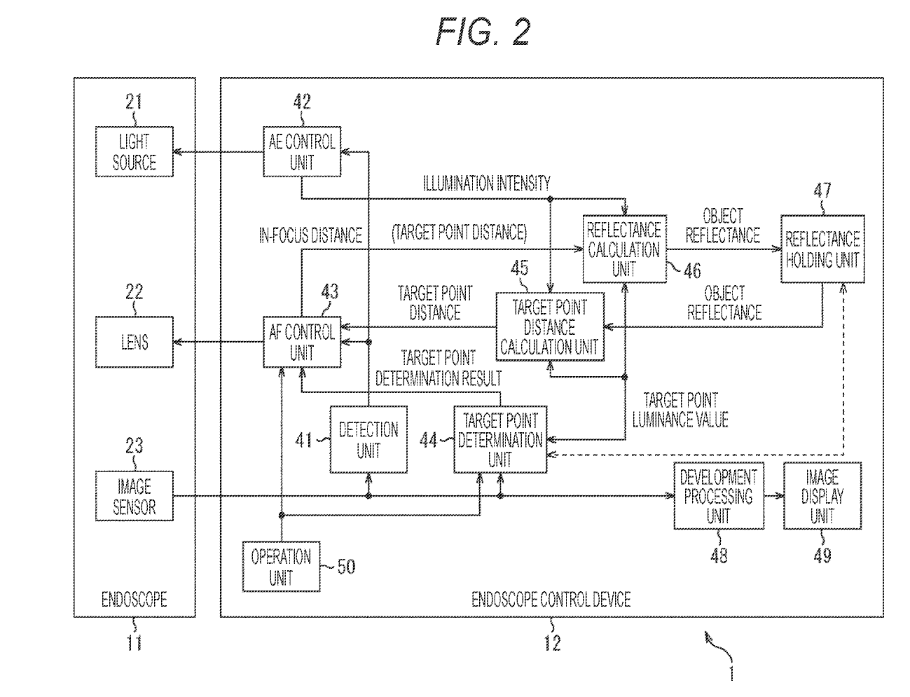

FIG. 2 is a block diagram showing example configurations of the endoscope and the endoscope control device. Note that, in the example in FIG. 2, the transmission unit, the reception unit, and the like between the endoscope and the endoscope control device are not shown, for ease of explanation.

In the example shown in FIG. 2, the endoscope 11 includes a light source 21, a lens 22, and an image sensor 23.

The endoscope control device 12 includes a detection unit 41, an automatic exposure (AE) control unit 42, an automatic focus (AF) control unit 43, a target point determination unit 44, a target point distance calculation unit 45, a reflectance calculation unit 46, a reflectance holding unit 47, a development processing unit 48, an image display unit 49, and an operation unit 50.

The light source 21 emits light onto the subject 3, under the control of the AE control unit 42. The lens 22 is driven and focused, under the control of the AF control unit 43. The image sensor 23 captures, through the lens 22, light reflected from the subject illuminated with the light source 21, and outputs pixel value (image) information to the detection unit 41, the target point determination unit 44, and the development processing unit 48.

From the pixel value information acquired from the image sensor 23, the detection unit 41 calculates the brightness of the entire screen, the magnitude of the contrast of the target point, and the like. The detection unit 41 supplies the calculated brightness of the entire screen to the AE control unit 42. The detection unit 41 supplies the calculated magnitude of the contrast of the target point to the AF control unit 43.

In accordance with the brightness of the entire screen and the like acquired from the detection unit 41, the AE control unit 42 adjusts the intensity of the light source 21 to an appropriate brightness. Note that, when focus is achieved, the AE control unit 42 records the illumination intensity.

In accordance with the target point contrast magnitude and the like acquired from the detection unit 41, the AF control unit 43 adjusts the lens 22 to change the focus position so that the contrast is maximized. Note that, when focus is achieved, the AF control unit 43 records the focusing distance. Upon receipt of the target point distance from the target point distance calculation unit 45, the AF control unit 43 also searches for the focus position, using the received target point distance as the initial value.

When AF is started, the target point determination unit 44 determines whether or not the target point to be focused on in the current operation is the same as the target point that was previously focused on. In the target point determination, it is possible to adopt a method of determining whether or not the target point is in the same color or has the same texture (pattern) as the previous one, for example. Note that the brightness changes, and therefore, the colors are not RGB, but hues or the like is used. Block matching may also be used.

In that case, various sizes may be adopted.

The target point determination unit 44 also calculates the luminance value of the target point, and, when focus is achieved, records the calculated luminance value (luminance information) of the target point. The luminance value of the target point may be the luminance value at one point at the center, or the average value in a certain area.

The target point distance calculation unit 45 acquires the illumination intensity from the AE control unit 42, acquires the target point luminance value from the target point determination unit 44, and acquires the object reflectance from the reflectance holding unit 47.

The target point distance calculation unit 45 then calculates (predicts) the distance to the target point, using the illumination intensity acquired from the AE control unit 42, the target point luminance value acquired from the target point determination unit 44, and the object reflectance acquired from the reflectance holding unit 47. In the calculation, the above equation (3) is used. The target point distance calculation unit 45 supplies the calculated target point distance to the AF control unit 43. Note that, although brightness varies at any time depending on the AE control unit 42, it is possible to calculate an approximate distance by acquiring information about the brightness, the reflectance of the target point, and the luminance information.

The reflectance calculation unit 46 acquires the illumination intensity from the AE control unit 42, acquires the focusing distance as the target point distance from the AF control unit 43, and acquires the target point luminance value from the target point determination unit 44. The reflectance calculation unit 46 calculates the object (subject) reflectance, using the target point distance acquired from the AF control unit 43, the illumination intensity acquired from the AE control unit 42, and the target point luminance value acquired from the target point determination unit 44. In the calculation, the above equation (2) is used. The calculated object reflectance is supplied to the reflectance holding unit 47.

The reflectance holding unit 47 records the object reflectance calculated by the reflectance calculation unit 46. Note that the object reflectance is not necessarily calculated in the first AF operation. Instead, at the time of object reflectance recording, or as indicated by the dashed lines in advance, the information about the target point (information about the hue or the texture, information about the pattern, or the like) acquired from the target point determination unit 44 may be associated with the object reflectance and be stored. The corresponding reflectance may be retrieved from the stored information serving as a database, and be used. Further, the reflectance to be associated and stored may be a reflectance relating to an organ (such as the heart, fat, muscles), an individual, or the like, for example. For example, it is also possible to detect an abnormal change in a normal cell in accordance with the reflectance, and issue an alert.

The development processing unit 48 converts the pixel value information acquired from the image sensor 23 into an image compatible with the image display unit 49, to which the converted image is output, and causes the image display unit 49 to display the converted image. The image display unit 49 displays the image output from the development processing unit 48.

The operation unit 50 supplies an operation signal corresponding to a user operation to each component as necessary. For example, when the user issues a contrast AF start instruction, the operation unit 50 supplies an operation signal corresponding to a contrast AF start to the AF control unit 43 and the target point determination unit 44. Note that, in the case of continuous AF, the AF automatically starts when the power is turned on, for example.

<Operation of the Endoscope System>

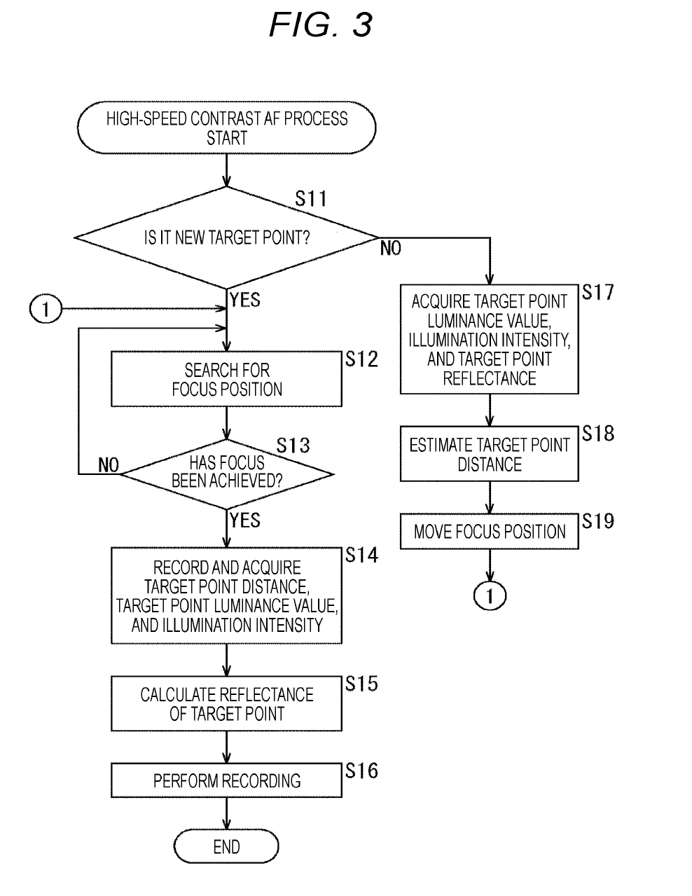

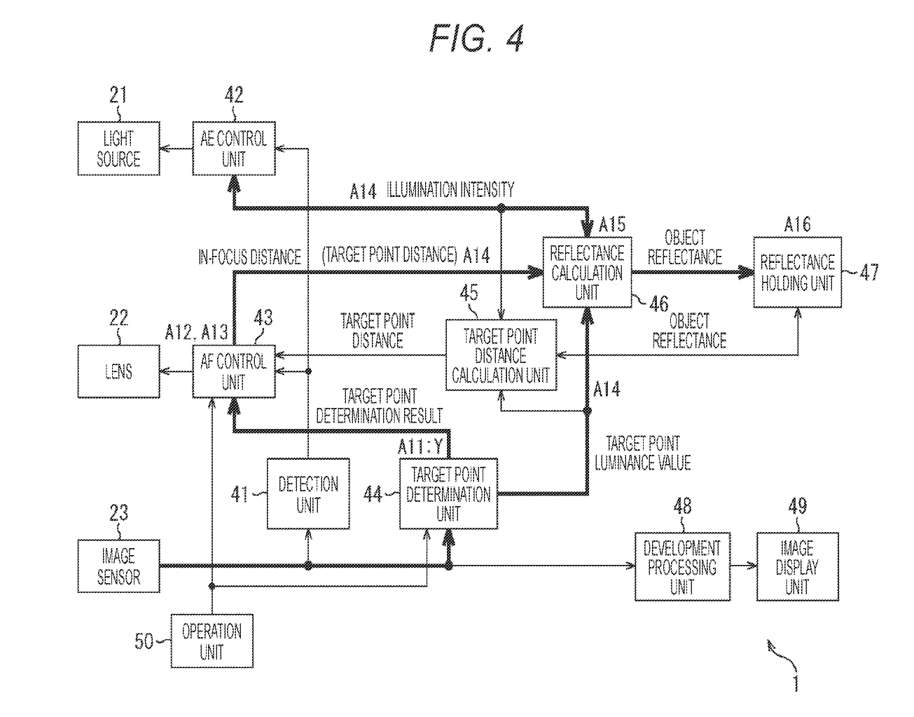

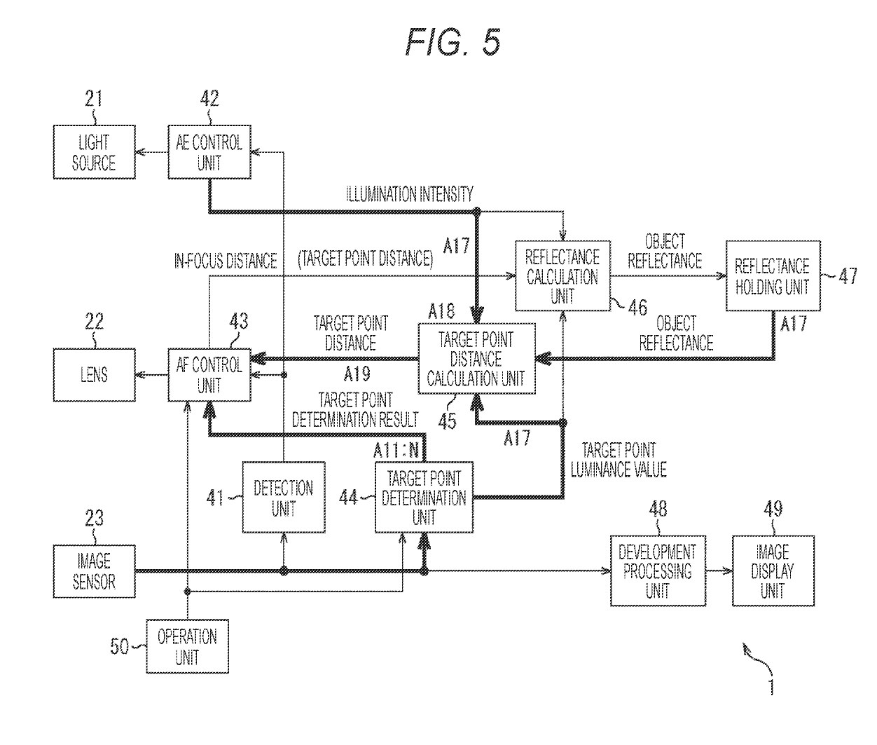

Referring now to the flowchart in FIG. 3, a high-speed contrast AF process to be performed by the endoscope system 1 is described. Note that contrast AF to which the present technology is applied will be hereinafter referred to as a high-speed contrast process, for ease of explanation. Further, the high-speed contrast process is divided into two stages of processes. FIG. 4 shows the flow of the process in the first stage. FIG. 5 shows the flow of the process in the second stage. Note that A11 through A19 shown in FIGS. 4 and 5 correspond to steps S11 through S19 in FIG. 3, respectively, and FIGS. 4 and 5 will be referred to in the description with reference to FIG. 3 as appropriate.

When the user issues a high-speed contrast AF start instruction via the operation unit 50, the operation unit 50 supplies an operation signal corresponding to a high-speed contrast AF start to the AF control unit 43 and the target point determination unit 44, and the process shown in FIG. 3 is started.

In step S11, the target point determination unit 44 determines whether or not the target point to be focused in the current operation is a new target point. In a case where the target point is determined to be a new target point in step S11 (Y in A11 in FIG. 4), the target point determination unit 44 supplies the result (YES) of the target point determination in step S11 to the AE control unit 43, and the process then moves on to step S12, which is the process in the first stage.

In steps S12 and S13, the AE control unit 43 performs a normal AF process. Specifically, the AE control unit 43 searches for the focus position in step S12 (A12 in FIG. 4), and determines whether or not focus has been achieved in step S13 (A13 in FIG. 4). In a case where it is determined in step S13 that focus has not been achieved, the process returns to step S12, and the procedures thereafter are repeated. In a case where it is determined in step S13 that focus has been achieved, the process moves on to step S14.

In step S14, the AF control unit 43 acquires and records the focal length, the target point determination unit 44 acquires and records the target point luminance, and the AE control unit 42 acquires and records the illumination intensity. The reflectance calculation unit 46 acquires the illumination intensity from the AE control unit 42, acquires the focusing distance as the target point distance from the AF control unit 43, and acquires the target point luminance value from the target point determination unit 44. (A14 in FIG. 4).

In step S15, the reflectance calculation unit 46 calculates the reflectance of the target point, using the target point distance acquired from the AF control unit 43, the illumination intensity acquired from the AE control unit 42, and the target point luminance value acquired from the target point determination unit 44 (A15 in FIG. 4).

In step S16, the reflectance calculation unit 46 records the calculated reflectance of the target point (A16 in FIG. 4), and the high-speed contrast AF process then comes to an end.

In a case where the target point is determined not to be a new target point in step S11, that is, the target point is the same as the previous one (N in A11 in FIG. 5), on the other hand, the target point determination unit 44 supplies the result (No) of the target point determination in step S11 to the AE control unit 43, and the process then moves on to step S17, which is the process in the second stage. In this case, the AE control unit 43 temporarily stops the normal AF operation.

In step S17, the target point distance calculation unit 45 acquires the illumination intensity from the AE control unit 42, acquires the target point luminance value from the target point determination unit 44, and acquires the object reflectance from the reflectance holding unit 47 (A17 in FIG. 5). In step S18, the target point distance calculation unit 45 then calculates (predicts) the target point distance to the target point, using the illumination intensity acquired from the AE control unit 42, the target point luminance value acquired from the target point determination unit 44, and the object reflectance acquired from the reflectance holding unit 47 (A18 in FIG. 5). In the calculation, the above equation (3) is used. The target point distance calculation unit 45 supplies the calculated target point distance to the AE control unit 43.

In step S19, after receiving the target point distance from the target point distance calculation unit 45, the AF control unit 43 moves the focus position to the received target point distance. After that, the process returns to step S12, and the AF control unit 43 repeats the normal contrast AF process, which is the subsequent process. In other words, after the target point distance is received, the focus position search in S12 is performed, with the target point distance being the initial value of the focus position. As a result, the contrast AF process speed can be increased in a case where the target point is the same as the previous one.

Note that it is also possible to further increase the accuracy by recording how accurately the distance information was calculated from the reflectance of the target point.

As described above, according to the present technology, it is possible to shorten the time before focusing, in a case where AF is performed again on a target point already subjected to an AF process once. Alternatively, it is possible to shorten the time before focusing, using an object reflectance database. This improves the usability of the endoscope system equipped with the AF.

Note that it is also possible to selectively use the high-speed contrast AF process of the present technology and the normal contrast AF process. Contrast AF is poor in contrast, and does not effectively work in the case of a blurred subject, resulting in a long time before focusing. To counter this, an example method of selective use may be a method of selectively using the high-speed contrast AF process of the present technology and the normal contrast AF process, in accordance with a contrast ratio threshold value and the contrast of an image of the subject.

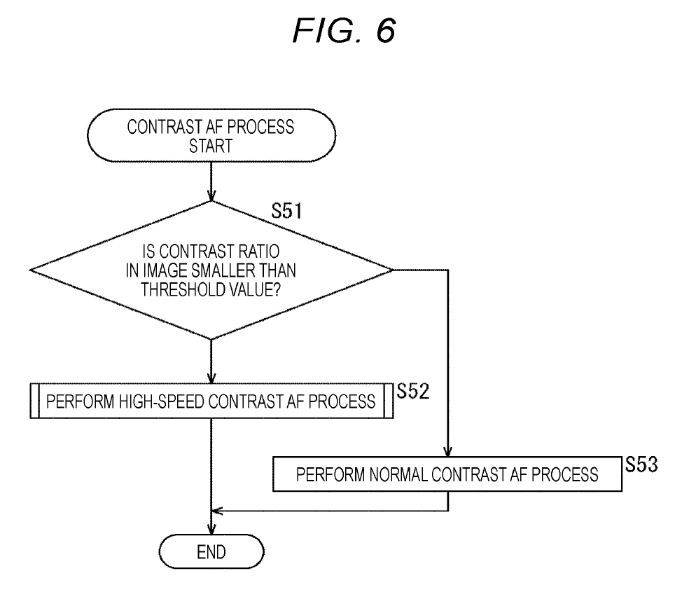

Next, a contrast AF process that is a process of selectively using the high-speed contrast AF process of the present technology and the normal contrast AF process is described, with reference to the flowchart in FIG. 6.

From the pixel value information acquired from the image sensor 23, the detection unit 41 calculates the brightness of the entire screen, the magnitude of the contrast of the target point, and the like. The detection unit 41 supplies the calculated magnitude of the contrast of the target point to the AF control unit 43.

In step S51, the AF control unit 43 determines whether or not the contrast ratio in the image is smaller than the threshold value. In a case where the contrast ratio in the image is determined to be lower than the threshold value in step S51, the process moves on to step S52. In step S52, the AF control unit 43 performs the high-speed contrast AF process described above with reference to FIG. 3.

In a case where the contrast ratio in the image is determined to be higher than the threshold value in step S51, on the other hand, the AF control unit 43 moves on to step S53. In step S53, the AF control unit 43 performs the normal contrast AF process (the process shown in steps S13 and S14 in FIG. 3 not using the initial value of the focus position like the present technology).

As described above, depending on the contrast ratio of the image, it is possible to use both the normal contrast AF process and the contrast AF process of the present technology.

Note that the present technology can be applied to any endoscope system of a rigid endoscope, a flexible endoscope (for surgeries, examinations, and the like), and an industrial endoscope. Further, the endoscope may use a single lens or a plurality of lenses such as two lenses, or may be an endoscope capable of stereoscopic viewing.

<Personal Computer>

The above described series of processes can be performed by hardware, or can be performed by software. In a case where the series of processes are to be performed by software, the program that forms the software is installed into a computer. Note that examples of the computer include a computer incorporated into dedicated hardware and a general-purpose personal computer or the like capable of executing various functions by installing various programs thereinto.

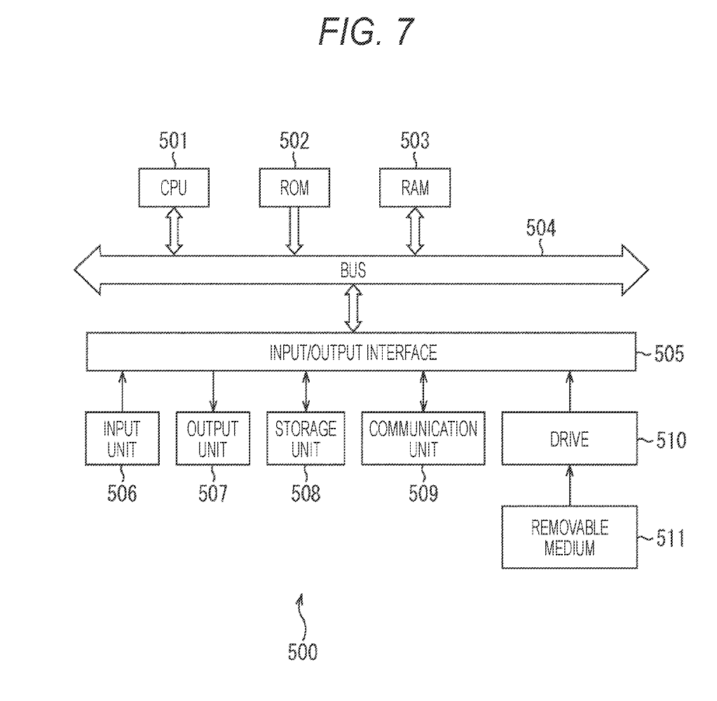

FIG. 7 is a block diagram showing an example configuration of the hardware of a personal computer that performs the above described series of processes in accordance with a program.

In the personal computer 500, a central processing unit (CPU) 501, a read only memory (ROM) 502, and a random access memory (RAM) 503 are connected to one another by a bus 504.

An input/output interface 505 is further connected to the bus 504. An input unit 506, an output unit 507, a storage unit 508, a communication unit 509, and a drive 510 are connected to the input/output interface 505.

The input unit 506 is formed with a keyboard, a mouse, a microphone, and the like. The output unit 507 is formed with a display, a speaker, and the like. The storage unit 508 is formed with a hard disk, a nonvolatile memory, or the like. The communication unit 509 is formed with a network interface or the like. The drive 510 drives a removable medium 511 such as a magnetic disk, an optical disk, a magnetooptical disk, or a semiconductor memory.

In the personal computer 500 having the above described configuration, the CPU 501 loads a program stored in the storage unit 508 into the RAM 503 via the input/output interface 505 and the bus 504, for example, and executes the program. As a result, the series of processes described above is performed.

The program to be executed by the computer (the CPU 501) can be recorded on the removable medium 511, and be provided. The removable medium 511 is a packaged medium or the like that is formed with a magnetic disk (including a flexible disk), an optical disk (such as a Compact Disc-read only memory (CD-ROM) or a digital versatile disc (DVD)), a magnetooptical disk, a semiconductor memory, or the like, for example. Alternatively, the program can be provided via a wired or wireless transmission medium, such as a local area network, the Internet, or digital satellite broadcasting.

In the computer, the program can be installed into the storage unit 508 via the input/output interface 505 when the removable medium 511 is mounted on the drive 510. Alternatively, the program may be received by the communication unit 509 through a wired or wireless transmission medium, and be installed into the storage unit 508. Other than the above, the program may be installed beforehand into the ROM 502 or the storage unit 508.

Note that the program to be executed by the computer may be a program for performing processes in chronological order in accordance with the sequence described in this specification, or may be a program for performing processes in parallel or performing a process in a necessary stage, such as when there is a call.

Also, in this specification, steps describing a program recorded on a recording medium include processes to be performed in parallel or independently of one another if not necessarily in chronological order, as well as processes to be performed in chronological order in accordance with the sequence described herein.

Further, in this specification, a system refers to an entire apparatus formed with a plurality of devices.

For example, the present disclosure can be embodied in a cloud computing configuration in which one function is shared among a plurality of devices via a network, and processing is performed by the devices cooperating with one another.

Note that the program to be executed by the computer may be a program for performing processes in chronological order in accordance with the sequence described in this specification, or may be a program for performing processes in parallel or performing a process in a necessary stage, such as when there is a call.

Also, in this specification, steps describing a program recorded on a recording medium include processes to be performed in parallel or independently of one another if not necessarily in chronological order, as well as processes to be performed in chronological order in accordance with the sequence described herein.

Further, in this specification, a system refers to an entire apparatus formed with a plurality of devices.

For example, the present disclosure can be embodied in a cloud computing configuration in which one function is shared among a plurality of devices via a network, and processing is performed by the devices cooperating with one another.

Furthermore, any configuration described above as one device (or processing unit) may be divided into a plurality of devices (or processing units). Conversely, any configuration described above as a plurality of devices (or processing units) may be combined into one device (or processing unit). Alternatively, it is of course possible to add components other than those described above to the configuration of any of the devices (or processing units). Further, some components of a device (or processing unit) may be incorporated into the configuration of another device (or processing unit) as long as the configuration and the functions of the entire system remain substantially the same. That is, the present technology is not limited to the embodiments described above, but various modifications may be made to them without departing from the scope of the technology.

While preferred embodiments of the present disclosure have been described above with reference to the accompanying drawings, the present disclosure is not limited to those examples. It is apparent that those who have ordinary skills in the art can make various changes or modifications within the scope of the technical spirit claimed herein, and it should be understood that those changes or modifications are within the technical scope of the present disclosure.

Note that the present technology may also be embodied in the configurations described below.

(1) A control device including:

a distance calculation unit that calculates a distance from a lens to a target point in an image, using luminance information about the target point, a reflectance of the target point, and an intensity of a light, the lens having a fixed positional relationship with the light; and

a focus control unit that searches for a focus position, with an initial value being the distance to the target point calculated by the distance calculation unit.

(2) The control device according to (1), further including

a target point determination unit that determines whether or not the target point is a new target point,

in which,

in a case where the target point determination unit determines that the target point is not a new target point,

the distance calculation unit calculates a distance to the target point, and

the focus control unit searches for the focus position, with the initial value being the distance to the target point calculated by the distance calculation unit.

(3) The control device according to (2), further including

a reflectance calculation unit that calculates the reflectance of the target point,

in which,

in a case where the target point determination unit determines that the target point is a new target point,

the focus control unit searches for the focus position to acquire a distance to the target point, and

the reflectance calculation unit calculates the reflectance of the target point, using the distance to the target point acquired by the focus control unit, the luminance information about the target point, and the intensity of the light.

(4) The control device according to (3), in which the reflectance of the target point calculated by the reflectance calculation unit is registered in association with target point information about the target point.

(5) The control device according to (4), in which the reflectance of the target point is registered in association with the target point information about the target point.

(6) The control device according to (4), in which the reflectance of the target point is registered for each organ.

(7) The control device according to (4), in which the reflectance of the target point is registered for each individual.

(8) The control device according to any of (2) to (7), in which the target point determination unit determines whether or not the target point is a new target point, by matching.

(9) The control device according to any of (1) to (8), further including

a contrast determination unit that determines whether or not a contrast ratio in the image is lower than a threshold value,

in which,

in a case where the contrast determination unit determines that the contrast ratio in the image is lower than the threshold value,

the distance calculation unit calculates a distance to the target point, and

the focus control unit searches for the focus position, with the initial value being the distance to the target point calculated by the distance calculation unit.

(10) The control device according to (9), in which,

in a case where the contrast determination unit determines that the contrast ratio in the image is higher than the threshold value,

the focus control unit searches for the focus position without the initial value.

(11) The control device according to any of (1) to (10), in which the lens and the light are provided in an endoscope.

(12) A control method implemented by a control device,

the control method including:

calculating a distance from a lens to a target point in an image, using luminance information about the target point, a reflectance of the target point, and an intensity of a light, the lens having a fixed positional relationship with the light; and

searching for a focus position, with an initial value being the calculated distance to the target point.

(13) A program for causing a computer to function as:

a distance calculation unit that calculates a distance from a lens to a target point in an image, using luminance information about the target point, a reflectance of the target point, and an intensity of a light, the lens having a fixed positional relationship with the light; and

a focus control unit that searches for a focus position, with an initial value being the distance to the target point calculated by the distance calculation unit.

(14) An endoscope system including:

an endoscope including:

a light that illuminates a subject;

a lens that has a fixed positional relationship with the light; and

an imaging unit that captures an image of a subject by capturing, via the lens, light reflected from the subject illuminated with the light; and

a control device including:

a distance calculation unit that calculates a distance from the lens to a target point, using luminance information about the target point in the image captured by the imaging unit, a reflectance of the target point, and an intensity of the light; and

a focus control unit that searches for a focus position, with an initial value being the distance to the target point calculated by the distance calculation unit.

### REFERENCE SIGNS LIST

1 Endoscope system
3 Subject
11 Endoscope
12 Endoscope control device
21 Light source
22 Lens
23 Image sensor
41 Detection unit
42 Automatic exposure (AE) control unit
43 Automatic focus (AF) control unit
44 Target point determination unit
45 Target point distance calculation unit
46 Reflectance calculation unit
47 Reflectance holding unit
48 Development processing unit
49 Image display unit
50 Operation unit

1. A control device comprising:
a distance calculation unit that calculates a distance from a lens to a target point in an image, using luminance information about the target point, a reflectance of the target point, and an intensity of a light, the lens having a fixed positional relationship with the light; and
a focus control unit that searches for a focus position, with an initial value being the distance to the target point calculated by the distance calculation unit.

2. The control device according to claim 1, further comprising
a target point determination unit that determines whether or not the target point is a new target point,
wherein,
in a case where the target point determination unit determines that the target point is not a new target point,
the distance calculation unit calculates a distance to the target point, and
the focus control unit searches for the focus position, with the initial value being the distance to the target point calculated by the distance calculation unit.

3. The control device according to claim 2, further comprising
a reflectance calculation unit that calculates a reflectance of the target point,
wherein,
in a case where the target point determination unit determines that the target point is a new target point,
the focus control unit searches for the focus position to acquire a distance to the target point, and
the reflectance calculation unit calculates the reflectance of the target point, using the distance to the target point acquired by the focus control unit, the luminance information about the target point, and the intensity of the light.

4. The control device according to claim 3, wherein the reflectance of the target point calculated by the reflectance calculation unit is registered in association with target point information about the target point.

5. The control device according to claim 1, wherein the reflectance of the target point is registered in association with target point information about the target point.

6. The control device according to claim 5, wherein the reflectance of the target point is registered for each organ.

7. The control device according to claim 5, wherein the reflectance of the target point is registered for each individual.

8. The control device according to claim 2, wherein the target point determination unit determines whether or not the target point is a new target point, by matching.

9. The control device according to claim 1, further comprising
a contrast determination unit that determines whether or not a contrast ratio in the image is lower than a threshold value,
wherein,
in a case where the contrast determination unit determines that the contrast ratio in the image is lower than the threshold value,
the distance calculation unit calculates a distance to the target point, and
the focus control unit searches for the focus position, with the initial value being the distance to the target point calculated by the distance calculation unit.

10. The control device according to claim 9, wherein,
in a case where the contrast determination unit determines that the contrast ratio in the image is higher than the threshold value,
the focus control unit searches for the focus position without an initial value.

11. The control device according to claim 1, wherein the lens and the light are provided in an endoscope.

12. A control method implemented by a control device,
the control method comprising:
calculating a distance from a lens to a target point in an image, using luminance information about the target point, a reflectance of the target point, and an intensity of a light, the lens having a fixed positional relationship with the light; and
searching for a focus position, with an initial value being the calculated distance to the target point.

13. A program for causing a computer to function as:
a distance calculation unit that calculates a distance from a lens to a target point in an image, using luminance information about the target point, a reflectance of the target point, and an intensity of a light, the lens having a fixed positional relationship with the light; and
a focus control unit that searches for a focus position, with an initial value being the distance to the target point calculated by the distance calculation unit.

14. An endoscope system comprising:
an endoscope including:
a light that illuminates a subject;
a lens that has a fixed positional relationship with the light; and
an imaging unit that captures an image of a subject by capturing, via the lens, light reflected from the subject illuminated with the light; and
a control device including:
a distance calculation unit that calculates a distance from the lens to a target point, using luminance information about the target point in the image captured by the imaging unit, a reflectance of the target point, and an intensity of the light; and
a focus control unit that searches for a focus position, with an initial value being the distance to the target point calculated by the distance calculation unit.
